(19) United States
(12) Patent Application Publication
(10) Pub. No.: US 2024/0409547 A1
(43) Pub. Date: Dec. 12, 2024

(54) Solid Forms of Upadacitinib

(71) Applicant: Apotex Inc., Toronto (CA)

(72) Inventors: Jeffrey S. Price, Brantford (CA); Fabio E. S. Souza, Brantford (CA); Allan W. Rey, Brantford (CA); Murali Kondamreddy, Brantford (CA)

(21) Appl. No.: 18/737,389

(22) Filed: Jun. 7, 2024

Related U.S. Application Data
(60) Provisional application No. 63/472,020, filed on Jun. 9, 2023.

(51) Int. Cl. C07D 487/14; A61K 31/4985

(52) U.S. Cl. CPC C07D 487/14; A61K 31/4985

(57) ABSTRACT

Provided are solid forms of upadacitinib. Specific solid forms include an amorphous solid dispersion of upadacitinib and magnesium chloride and crystalline forms of upadacitinib incorporating magnesium chloride, magnesium acetate, magnesium orotate, magnesium fumarate, magnesium citrate, or orotic acid. Also provided are pharmaceutical compositions including the upadacitinib solid forms and the use of these forms in the treatment of rheumatoid arthritis, psoriatic arthritis, atopic dermatitis, ulcerative colitis, and/or ankylosing spondylitis.

### CROSS-REFERENCE TO RELATED APPLICATION

This application claims priority to U.S. Provisional Patent Application No. 63/472,020 filed Jun. 9, 2023, the disclosure of which is hereby incorporated by reference in its entirety.

### BACKGROUND

### Technical Field

The present disclosure is directed to solid forms of upadacitinib, pharmaceutical compositions comprising these forms, and their use in the treatment of Janus kinase (JAK)-associated conditions comprising rheumatoid arthritis, psoriatic arthritis, atopic dermatitis, ulcerative colitis, and/or ankylosing spondylitis.

### Technical Considerations

Upadacitinib (1), or (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide is the active pharmaceutical ingredient (API), in the form of the hemihydrate, in RINVOQ®, a prescription medication for use in the treatment of patients with rheumatoid arthritis, psoriatic arthritis, atopic dermatitis, ulcerative colitis, and/or ankylosing spondylitis, subject to criteria set forth on labeling.

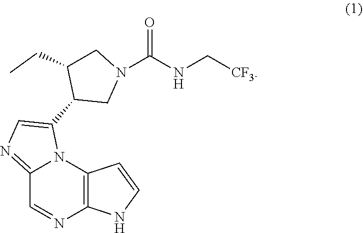 [SMILES: CC[C@@H]1CN(C(=O)NCC(F)(F)F)C[C@@H]1c1cnc2cnc3[nH]ccc3n12]

Amorphous and crystalline solid forms of upadacitinib, including hydrated, solvated, co-crystal, and/or salt forms thereof, are reported in, for example, WO 2017/066775 A1, US 2022/0002306 A1, WO 2020/115212 A1, WO 2020/115213 A1, US 2021/0380596 A1, US 2022/0041611 A1, WO 2021/244323 A1, IN 202041025283, U.S. Pat. No. 11,572,365 B2, CN 112110929 A, IN 202041052794, WO 2022/217257 A1, and IN 202241056709.

According to WO 2017/066775 A1, a concern with certain extended-release tablets comprising upadacitinib free base hydrate Form C is that they exhibit a slower release of upadacitinib at higher pH (6.8) compared to low pH (0.1 N HCl). In contrast, compositions incorporating an organic acid as a pH modifier, such as tartaric acid, are described in WO 2017/066775 A1, which exhibit pH-independent release.

Different solid forms of the same compound may have different packing, thermodynamic, spectroscopic, kinetic, surface, and mechanical properties. For example, a particular solid form of a compound which incorporates a co-former may exhibit a modified and/or stabilized dissolution profile, including over a range of pH conditions, in comparison to a different solid form of the same compound. Particular solid forms may also have different dissolution rates, thereby providing different pharmacokinetic parameters, which allow for specific forms to be used in order to achieve specific pharmacokinetic targets. Different solid forms may have different stability properties. A particular solid form may be more sensitive to heat, relative humidity (RH), and/or light. Alternatively, or additionally, a particular solid form may have different compressibility and/or density properties thereby providing more desirable characteristics for formulation and/or product manufacturing. Additionally, the particular solubility characteristics of a given solid form in relation to undesired impurities can result in differences in the chemical purity of different salt and/or crystalline forms upon isolation. Differences in stability may result from changes in chemical reactivity, such as differential oxidation. Such properties may provide for more suitable product qualities, such as a dosage form that is more resistant to discolouration when comprised of a specific solid form. Different physical properties of solid forms may also affect their processing. For example, a particular solid form may be more cohesive, more resistant to flow, less capable of dispersing static charge, or may be more difficult to filter and/or wash.

There exists a need for novel solid forms of upadacitinib having improved properties for use in providing drug products comprising upadacitinib.

### SUMMARY

The present disclosure provides upadacitinib solid forms comprising upadacitinib and a magnesium salt selected from the group consisting of magnesium chloride, magnesium acetate, magnesium orotate, magnesium fumarate, and magnesium citrate. Further provided by the present disclosure are crystalline forms comprising upadacitinib and orotic acid, a pharmaceutically acceptable acid. It is expected that the magnesium salts of the present disclosure, which further comprise pharmaceutically acceptable counter-ions, can be safely used in materials intended for use in the preparation of pharmaceutical compositions intended for administration to humans or animals.

Accordingly, in a first embodiment of the present disclosure, there is provided a crystalline form of upadacitinib comprising upadacitinib and a magnesium salt. In some embodiments, the magnesium salt is selected from the group consisting of magnesium chloride, magnesium acetate, magnesium orotate, magnesium fumarate and magnesium citrate. In some embodiments, the molar ratio of upadacitinib to the magnesium salt is from about 1:0.4 to about 1:5. In some embodiments, the crystalline form is a hydrate. In some embodiments, the crystalline form is anhydrous.

In a second embodiment of the present disclosure, there is provided a crystalline form of upadacitinib comprising upadacitinib and magnesium chloride. In some embodiments, the molar ratio of upadacitinib to magnesium chloride is from about 1:0.4 to about 1:1. In some embodiments, the molar ratio of upadacitinib to magnesium chloride is about 1:0.4 to about 1:0.6. In some embodiments, the crystalline form is a hydrate. In some embodiments, the crystalline form is characterized by a PXRD diffractogram comprising peaks, expressed in degrees 2θ(±0.2°), at 4.3°, 5.5°, and 21.1°. In some embodiments, the crystalline form is characterized by a PXRD diffractogram further comprising at least three peaks, expressed in degrees 2θ (±0.2°), selected from the group consisting of: 8.1°, 8.7°, 13.3°, 15.7°, 17.1°, and 23.6°. In some embodiments, the PXRD diffractogram further comprises peaks, expressed in degrees 2θ (±0.2°), at 8.10, 8.7°, 13.3°, 15.7°, 17.1°, and 23.6°. In some embodiments, the crystalline form provides a PXRD diffractogram comprising peaks in substantially the same positions (±0.2° 2θ) as those shown in FIG. 1. In some embodiments, the crystalline form provides a PXRD diffractogram comprising peaks in substantially the same positions (±0.2°2θ) as those shown in FIG. 9.

In a third embodiment of the present disclosure, there is provided a crystalline form of upadacitinib comprising upadacitinib and magnesium acetate. In some embodiments, the molar ratio of upadacitinib to the magnesium acetate is about 1:0.4 to about 1:5. In some embodiments, the crystalline form is characterized by a PXRD diffractogram comprising peaks, expressed in degrees 2θ (±0.2°), at 7.6°, 11.0°, and 21.7°. In some embodiments, the crystalline form is characterized by a PXRD diffractogram further comprising at least three peaks, expressed in degrees 2θ (±0.2°), selected from the group consisting of: 7.9°, 15.8°, 17.0°, 17.6°, 20.3°, and 26.6°. In some embodiments, the PXRD diffractogram further comprises peaks, expressed in degrees 2θ (±0.2°), at: 7.9°, 15.8°, 17.0°, 17.6°, 20.3°, and 26.6°. In some embodiments, the crystalline form provides a PXRD diffractogram comprising peaks in substantially the same positions (±0.2°2θ) as those shown in FIG. 2.

In a fourth embodiment of the present disclosure, there is provided a crystalline form of upadacitinib comprising upadacitinib and magnesium orotate. In some embodiments, the crystalline form is characterized by a PXRD diffractogram comprising peaks, expressed in degrees 2θ (±0.2°), at 8.1°, 10.6°, and 28.6°. In some embodiments, the crystalline form is characterized by a PXRD diffractogram further comprising at least three peaks, expressed in degrees 2θ (±0.2°), selected from the group consisting of: 14.6°, 16.3°, 17.7°, 21.4°, 22.2°, and 26.0°. In some embodiments, the PXRD diffractogram further comprises peaks, expressed in degrees 2θ (±0.2°), at: 14.6°, 16.3°, 17.7°, 21.4°, 22.2°, and 26.0°. In some embodiments, the crystalline form provides a PXRD diffractogram comprising peaks in substantially the same positions (±0.2°2θ) as those shown in FIG. 6.

In a fifth embodiment of the present disclosure, there is provided a crystalline form of upadacitinib comprising upadacitinib and magnesium fumarate. In some embodiments, the crystalline form is characterized by a PXRD diffractogram comprising peaks, expressed in degrees 2θ (±0.2°), 9.1°, 11.8°, and 26.6°. In some embodiments, the crystalline form is characterized by a PXRD diffractogram further comprising at least three peaks, expressed in degrees 2θ (±0.2°), selected from the group consisting of: 12.4°, 16.1°, 17.1°, 18.3°, 21.7°, and 22.6°. In some embodiments, the PXRD diffractogram further comprises peaks, expressed in degrees 2θ (±0.2°), at: 12.4°, 16.1°, 17.1°, 18.3°, 21.7°, and 22.6°. In some embodiments, the crystalline form provides a PXRD diffractogram comprising peaks in substantially the same positions (±0.2°2θ) as those shown in FIG. 7.

In a sixth embodiment of the present disclosure, there is provided a crystalline form of upadacitinib comprising upadacitinib and orotic acid. In some embodiments, the molar ratio of upadacitinib to orotic acid is about 1:2. In some embodiments, the crystalline form is characterized by a PXRD diffractogram comprising peaks, expressed in degrees 2θ (±0.2°), 5.3°, 12.0°, and 17.7°. In some embodiments, the crystalline form is characterized by a PXRD diffractogram further comprising at least three peaks, expressed in degrees 2θ (±0.2°), selected from the group consisting of: 8.8°, 10.6°, 12.9°, 14.6°, 15.9°, and 16.7°. In some embodiments, the PXRD diffractogram further comprises peaks, expressed in degrees 2θ (±0.2°), at: 8.8°, 10.6°, 12.9°, 14.6°, 15.9°, and 16.7°. In some embodiments, the crystalline form provides a PXRD diffractogram comprising peaks in substantially the same positions (±0.2°2θ) as those shown in FIG. 4. In some embodiments, the crystalline form is characterized by a PXRD diffractogram comprising peaks, expressed in degrees 2θ (±0.2°), 4.7°, 9.5°, and 14.2°. In some embodiments, the crystalline form is characterized by a PXRD diffractogram further comprising at least three peaks, expressed in degrees 2θ (±0.2°), selected from the group consisting of: 11.2°, 11.8°, 15.3°, 16.0°, 19.0°, and 20.5°. In some embodiments, the PXRD diffractogram further comprises peaks, expressed in degrees 2θ (±0.2°), at: 11.2°, 11.8°, 15.3°, 16.0°, 19.0°, and 20.5°. In some embodiments, the crystalline form provides a PXRD diffractogram comprising peaks in substantially the same positions (±0.2° 2θ) as those shown in FIG. 5.

In a seventh embodiment of the present disclosure, there is provided an amorphous solid dispersion of upadacitinib comprising upadacitinib and a magnesium salt. In some embodiments, the molar ratio of upadacitinib to the magnesium salt is from about 1:0.4 to about 1:1. In some embodiments, the molar ratio of upadacitinib to the magnesium salt is from about 1:0.4 to about 1:0.6. In some embodiments, in the amorphous solid dispersion, the magnesium salt is magnesium chloride.

In an eighth embodiment of the present disclosure, there is provided a pharmaceutical composition comprising a crystalline form of upadacitinib according to the first, second, third, fourth, fifth or sixth embodiments disclosed herein, or the amorphous solid dispersion of the seventh embodiment disclosed herein, and one or more pharmaceutically acceptable excipients. In some embodiments, the pharmaceutical composition is in the form of a capsule or a tablet. In some embodiments, the pharmaceutical composition of the eighth embodiment is an extended-release tablet that comprises an amount of the upadacitinib crystalline form of the first, second, third, fourth, fifth or sixth embodiments, or the amorphous solid dispersion of the seventh embodiment of the disclosure, that is equivalent to 15 mg of anhydrous upadacitinib.

In a ninth embodiment of the present disclosure, there is provided the use of a crystalline form of upadacitinib according to the first, second, third, fourth, fifth or sixth embodiments, or the amorphous solid dispersion of the seventh embodiment of the disclosure, in the treatment of a Janus kinase (JAK)-associated condition. In some embodiments, the JAK-associated condition is selected from the group consisting of rheumatoid arthritis, psoriatic arthritis, atopic dermatitis, ulcerative colitis, and ankylosing spondylitis.

In an tenth embodiment of the present disclosure, there is provided a method of treating a Janus kinase (JAK)-associated condition, such as rheumatoid arthritis, psoriatic arthritis, atopic dermatitis, ulcerative colitis, or ankylosing spondylitis, comprising administering to a human subject in need thereof a therapeutically effective amount of crystalline form according to the first, second, third, fourth, fifth or sixth embodiments, or the amorphous solid dispersion of the seventh embodiment of the disclosure, or a combination thereof, or the pharmaceutical composition of the ninth embodiment of the disclosure.

Other embodiments and features of the present disclosure will become apparent to those ordinarily skilled in the art upon review of the following description of specific non-limiting embodiments of the disclosure in conjunction with the accompanying figures.

### BRIEF DESCRIPTION OF THE DRAWINGS

The terms FIG., FIGS., Figure, and Figures are used interchangeably in the specification to refer to the corresponding figures in the drawings. Non-limiting embodiments of the present disclosure are described, by way of example only, with reference to the attached drawings.

FIG. 1 is a representative PXRD diffractogram of upadacitinib Form APO-I as prepared in Example 1.

FIG. 2 is a representative PXRD diffractogram of upadacitinib Form APO-II as prepared in Example 2.

FIG. 3 is a representative PXRD diffractogram of upadacitinib Form APO-III as prepared in Example 3.

FIG. 4 is a representative PXRD diffractogram of upadacitinib Form APO-IV as prepared in Example 4.

FIG. 5 is a representative PXRD diffractogram of upadacitinib Form APO-V as prepared in Example 5.

FIG. 6 is a representative PXRD diffractogram of upadacitinib Form APO-VI as prepared in Example 6.

FIG. 7 is a representative PXRD diffractogram of upadacitinib Form APO-VII as prepared in Example 7.

FIG. 8 is a representative PXRD diffractogram of a mixture of upadacitinib Form APO-VIII and crystalline trimagnesium dicitrate polyhydrate as prepared in Example 8.

FIG. 9 is a representative PXRD diffractogram of upadacitinib Form APO-I as prepared in Example 11.

FIG. 10 is a reference PXRD diffractogram of crystalline trimagnesium dicitrate polyhydrate.

### DETAILED DESCRIPTION

The present disclosure provides upadacitinib solid forms comprising upadacitinib together with a magnesium salt selected from one of magnesium chloride, magnesium acetate, magnesium orotate, magnesium fumarate, and magnesium citrate. Further provided are crystalline forms comprising upadacitinib and orotic acid.

The upadacitinib solid forms of the present disclosure exhibit differences in properties when compared to the known solid forms of upadacitinib. Properties that differ between the disclosure and known solid forms of upadacitinib include, but are not limited to, crystal packing properties such as molar volume, density and/or hygroscopicity; thermodynamic properties such as melting point and/or solubility; kinetic properties such as dissolution rate and/or chemical/polymorphic stability; surface properties such as crystal habit/particle morphology; and/or mechanical properties such as hardness, tensile strength, compactibility, tabletting, handling, flow, and/or blending.

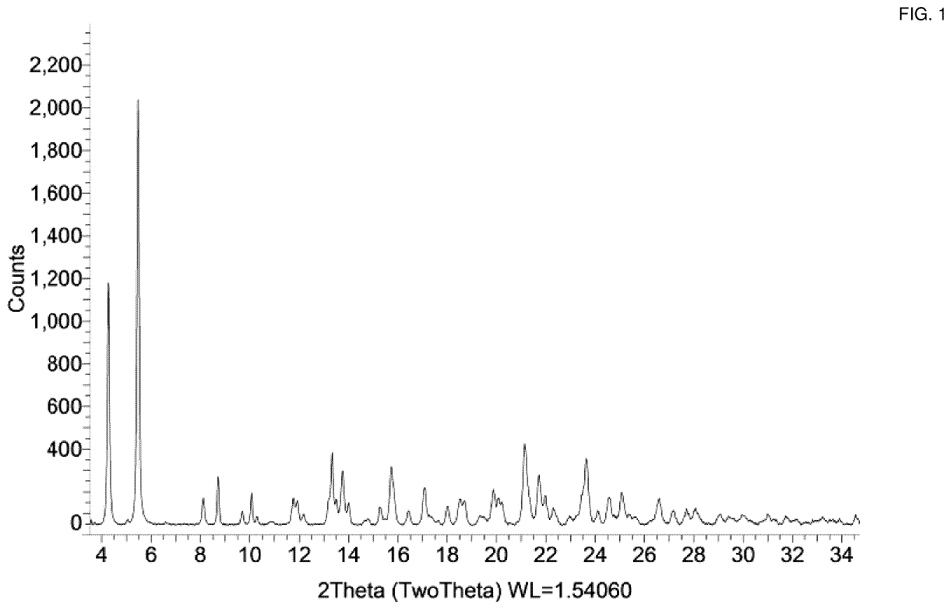

Depending on the manner in which the solid forms of the present disclosure are prepared, and the methodology and instrument used for PXRD analysis, the intensity of a given peak observed in a PXRD diffractogram of the solid form may vary when compared to the same peak in the representative PXRD diffractograms provided in FIGS. 1 to 9. Thus, differences in relative peak intensities between peaks in a PXRD diffractogram for a given solid form may be observed when compared to the relative peak intensities of the peaks in the representative PXRD diffractograms of FIGS. 1 to 8. Any such differences may be due, in part, to the preferred orientation of the sample and its deviation from the ideal random sample orientation, the preparation of the sample for analysis, and the methodology applied for the analysis. Such variations are known and understood by a person of skill in the art, and any such variations do not depart from the disclosure herein.

In addition to the differences in relative peak intensities that may be observed in comparison to the representative PXRD diffractograms provided in FIGS. 1 to 9, it is understood that individual peak positions may vary between ±0.2° 2θ from the values observed in the representative PXRD diffractograms provided in FIGS. 1 to 9 for the solid forms of the disclosure or listed in Tables 1 to 7. Such variations are known and understood by a person of skill in the art, and any such variations do not depart from the disclosure herein.

Further, depending on the instrument used for X-ray analysis and its calibration, uniform offsets in the peak position of each peak in a PXRD diffractogram of greater that 0.2° 2θ may be observed when compared to the representative PXRD diffractograms provided in FIGS. 1 to 9. Thus, PXRD diffractograms of the solid forms of the present disclosure may, in some circumstances, display the same relative peak positions as observed in the representative PXRD diffractograms provided in FIGS. 1 to 9, with the exception that each peak is offset in the same direction, and by approximately the same amount, such that the overall PXRD diffractogram is substantially the same in appearance as the PXRD diffractograms of FIGS. 1 to 9, with the exception of the uniform offset in peak positions. The observation of any such uniform peak shift in a PXRD diffractogram does not depart from the disclosure herein given that the relative peak positions of the individual peaks within the PXRD diffractogram remain consistent with the relative peak positions observed in the PXRD diffractograms of FIGS. 1 to 9.

As used herein, the singular form of “a”, “an”, and “the” include plural referents unless the context clearly states otherwise. As used herein, the term “solid form” refers to both crystalline forms and/or amorphous forms and/or dispersions thereof. As used herein, the term “crystalline form” is intended to include single-component and/or multiple-component crystalline forms. Single-component crystalline forms of upadacitinib consist solely of upadacitinib in the repeating unit of the crystal lattice. Multiple-component crystalline forms of upadacitinib include crystalline forms of upadacitinib wherein one or more other molecules, including for example solvent, water, and/or a co-former, are also incorporated into the crystal lattice with upadacitinib. Crystalline forms can be identified by physical characterization methods as described herein, such as PXRD, NMR, and/or Karl Fischer titration.

Multi-component crystalline forms comprising more than one type of molecule in the crystalline lattice may have some variability in the exact molar ratio of their components depending on the conditions used for their preparation. For example, a molar ratio of components within a multi-component crystalline form provides a person of skill in the art information as to the general relative quantities of the components of the crystalline form. In many cases, the molar ratio may vary by ±25% from a stated range. With respect to the present disclosure, a molar ratio of 1:0.4 should be understood to include the ratios 1:0.3 and 1:0.5, as well as all of the individual ratios in between.

As used herein, the term “room temperature” refers to a temperature of from about 20° C. to about 25° C.

Unless defined otherwise herein, the term “about”, when used in reference to molar ratios, allows for a variance of plus or minus 10%, or plus or minus 5%, or plus or minus 1% of the stated value. It should be understood that any numerical range recited herein is intended to include all sub-ranges subsumed therein.

As used herein, “magnesium chloride” refers to the salt of Formula MgCl₂, “magnesium acetate” refers to the salt of Formula Mg(CH₃CO₂)₂, “magnesium orotate” refers to the salt of Formula Mg(orotate)₂, “magnesium fumarate” refers to the salt of Formula Mg(CO₂CHCHCO₂), and “magnesium citrate” refers to the salt of Formula Mg(CO₂CH₂CO₂COHCH₂CO₂H).

As used herein, an “extended-release” (ER) dosage form refers to a drug product formulated in a manner that makes the drug substance available over an extended period of time following administration, compared to that observed or anticipated for an immediate-release dosage form. For example, the pharmaceutical compositions of the present disclosure can be provided as extended-release solid oral tablet dosage forms.

As used herein, chromatographic purity, expressed in area %, is determined by area normalisation of an high-performance liquid chromatography (HPLC) profile obtained using a method as disclosed herein. The purity of a sample is determined based on the ratio of the peak area of the analyte, such as a upadacitinib or an impurity of Formula UPA-III, to the total peak area of all components in the sample.

As used herein, the phrase “therapeutically effective amount” means that amount of solid form of upadacitinib that will elicit a biological or medical response of a tissue, system, or patient that is being sought by the administrator (such as a researcher, doctor, or veterinarian) which includes alleviation of the symptoms of the condition or disease being treated and the prevention, slowing, or halting of progression of the condition or disease, including but not limited to a Janus kinase (JAK)-associated condition, such as rheumatoid arthritis, psoriatic arthritis, atopic dermatitis, ulcerative colitis, and/or ankylosing spondylitis. In some examples, the pharmaceutical preparation is in a unit dosage form. In such form, the preparation is subdivided into suitably sized unit doses containing appropriate quantities of the active component, e.g., an effective amount to achieve the desired purpose. For convenience, the total daily dosage may be divided and administered in portions during the day as required. In some examples, the dosage can range from about 0.01 mg/kg to about 10 mg/kg of body weight/day of solid form of upadacitinib. It should be understood that any numerical range recited herein is intended to include all sub-ranges subsumed therein.

When describing the embodiments of the present disclosure there may be a common variance to a given temperature or time that would be understood or expected by the person skilled in the art to provide substantially the same result. For example, when reference is made to a particular temperature, it is to be understood by the person skilled in the art that there is an allowable variance of ±5° C. associated with that temperature. When reference is made to a particular time, it is to be understood that there is an allowable variance of ±10 minutes when the time is one or two hours, and ±1 hour when longer periods of time are referenced.

In one embodiment of the present disclosure, there is provided a new crystalline form of upadacitinib, upadacitinib Form APO-I, a magnesium chloride co-crystal. Preferably, in upadacitinib Form APO-I, the molar ratio of upadacitinib to magnesium chloride is about 1:0.5.

Upadacitinib Form APO-I can be characterized by a PXRD diffractogram comprising, among other peaks, characteristic peaks, expressed in degrees 2θ (±0.2°), at 4.3°, 5.5°, and 21.1°. In some embodiments, the PXRD diffractogram further comprises at least three peaks, expressed in degrees 2θ (±0.2°), selected from the group consisting of 8.1°, 8.7°, 13.3°, 15.7°, 17.1°, and 23.6°. In some embodiments, the PXRD diffractogram further comprises peaks, expressed in degrees 2θ (±0.2°), at 8.10, 8.7°, 13.3°, 15.7°, 17.1°, and 23.6°.

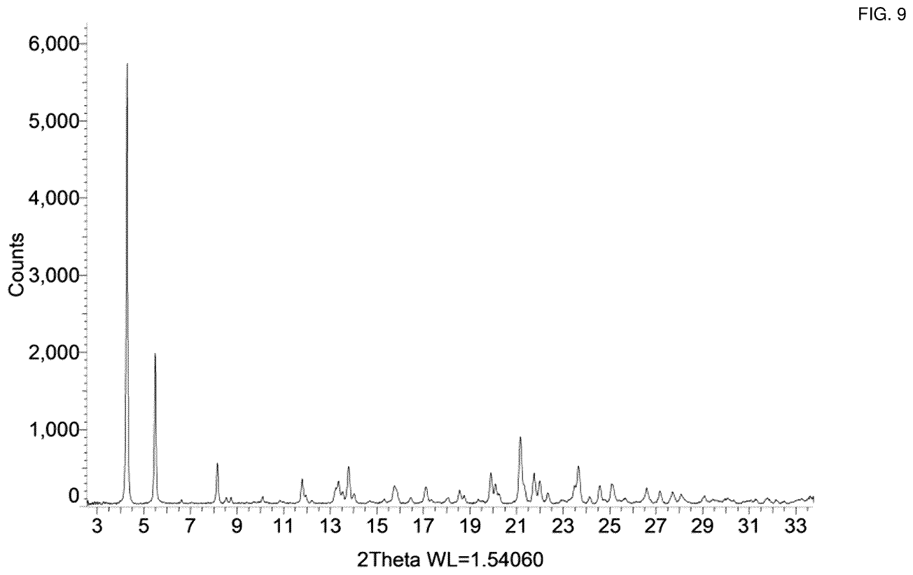

An illustrative PXRD diffractogram of upadacitinib Form APO-I, as prepared in Example 1, is shown in FIG. 1. A further illustrative PXRD diffractogram of upadactinib Form APO-I, as prepared in Example 11, is shown in FIG. 9. A peak listing, comprising representative peaks from the PXRD diffractogram in FIG. 1, and their relative intensities, is provided in Table 1. Although illustrative of the PXRD diffractogram that is provided for the upadacitinib Form APO-I of the present disclosure, the relative intensities of the peaks are variable. Thus, depending on a particular sample, the prominence or relative intensity of the peaks observed may differ from those in the illustrative PXRD diffractogram and peak listing.

TABLE 1
Relative Peak Intensities of Upadacitinib
Form APO-I from FIG. 1

| | Relative |
|---|---|
| Angle (2θ) | intensity (%) |
| 4.27 | 56.4 |
| 5.47 | 100.0 |
| 8.13 | 6.3 |
| 8.73 | 11.3 |
| 10.08 | 7.5 |
| 11.78 | 5.8 |
| 13.34 | 17.7 |
| 13.77 | 13.0 |
| 15.74 | 13.9 |
| 17.10 | 8.2 |
| 18.52 | 6.2 |
| 19.88 | 8.9 |
| 21.14 | 19.9 |
| 21.72 | 12.0 |
| 23.63 | 16.2 |
| 24.58 | 5.8 |
| 25.07 | 8.1 |

As described in Examples 1, 9 and 10, upadacitinib Form APO-I can be prepared by combining upadacitinib with magnesium chloride, in some embodiments about hemimolar amounts of anhydrous magnesium chloride, in a suitable solvent, preferably affording a solution. In some embodiments, the suitable solvent is selected from the group consisting of a C1-C3 alcohol, aqueous C1-C3 alcohol, a cyclic ether and/or mixtures thereof, for example a mixture of aqueous ethanol and 2-methyltetrahydrofuran. In some embodiments, a suspension is formed by addition of a suitable anti-solvent, preferably selected from the group consisting of ketones (e.g., acetone, methyl isobutyl ketone), esters (e.g. ethyl acetate, methyl acetate), ethers (e.g., methyl t-butyl ether, cyclopentyl methyl ether) and/or mixtures thereof. In some embodiments, the anti-solvent is ethyl acetate. After applying methods, if necessary, such as sonication, stirring, and/or heat cycling between about 0° C. and about 60° C. for a suitable time, the resulting suspension is isolated and dried, if necessary, preferably in vacuo, to afford upadacitinib Form APO-I having a PXRD diffractogram consistent with FIG. 1.

In a second embodiment of the present disclosure, there is provided a new crystalline form of upadacitinib, upadacitinib Form APO-II, comprising upadacitinib and magnesium acetate. In an embodiment, upadacitinib Form APO-II, is a magnesium acetate co-crystal.

Upadacitinib Form APO-II can be characterized by a PXRD diffractogram comprising, among other peaks, characteristic peaks, expressed in degrees 2θ (±0.2°), at 7.6°, 11.0°, and 21.7°. In some embodiments, the PXRD diffractogram further comprises at least three peaks, expressed in degrees 2θ (±0.2°), selected from the group consisting of 7.9°, 15.8°, 17.0°, 17.6°, 20.3°, and 26.6°. In some embodiments, the PXRD diffractogram further comprises peaks, expressed in degrees 2θ (±0.2°), at 7.9°, 15.8°, 17.0°, 17.6°, 20.3°, and 26.6°.

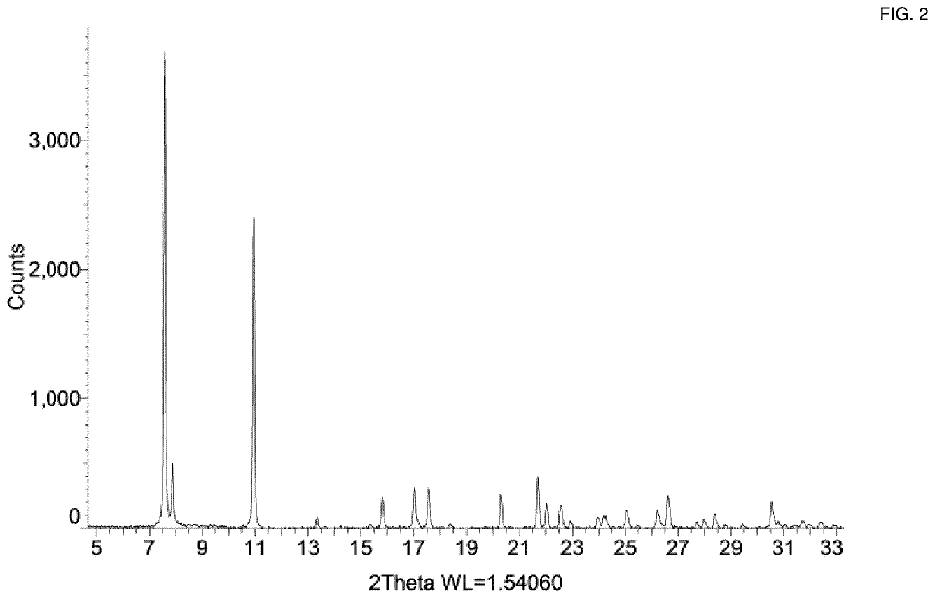

An illustrative PXRD diffractogram of upadacitinib Form APO-II, as prepared in Example 2, is shown in FIG. 2. A peak listing, comprising representative peaks from the PXRD diffractogram in FIG. 2, and their relative intensities, is provided in Table 2. Although illustrative of the PXRD diffractogram that is provided for the upadacitinib Form APO-II of the present disclosure, the relative intensities of the peaks are variable. Thus, depending on a particular sample, the prominence or relative intensity of the peaks observed may differ from those in the illustrative PXRD diffractogram and peak listing.

TABLE 2
Relative Peak Intensities of Upadacitinib
Form APO-II from FIG. 2

| Angle (2θ) | Relative intensity (%) |
|---|---|
| 7.59 | 100.0 |
| 7.89 | 13.5 |
| 10.95 | 67.1 |
| 15.81 | 6.9 |
| 17.03 | 8.9 |
| 17.57 | 8.4 |
| 20.30 | 9.0 |
| 21.70 | 13.4 |
| 22.03 | 6.3 |
| 22.57 | 5.3 |
| 25.04 | 4.9 |
| 26.20 | 4.9 |
| 26.62 | 8.2 |
| 30.54 | 7.3 |

As described in Example 2, upadacitinib Form APO-II can be prepared by combining upadacitinib with magnesium acetate, in some embodiments about equimolar amounts of magnesium acetate tetrahydrate, in a suitable solvent, preferably affording a solution. In some embodiments, the suitable solvent is a C1-C3 alcohol, preferably a mixture of methanol and trifluoroethanol, which is evaporated to dryness, and the resulting sample treated with a suitable anti-solvent, preferably ethyl acetate. After applying methods, if necessary, such as sonication, stirring, and/or heat cycling between about 0° C. and about 60° C. for a suitable time, the resulting suspension is isolated and dried, if necessary, preferably in vacuo, to afford upadacitinib Form APO-II having a PXRD diffractogram consistent with FIG. 2.

In a third embodiment of the present disclosure, there is provided a upadacitinib solid form, upadacitinib Form APO-III, an amorphous solid dispersion comprising upadacitinib and magnesium chloride. In some embodiments, in upadacitinib Form APO-Ill, the molar ratio of upadacitinib to magnesium chloride is about 1:0.5.

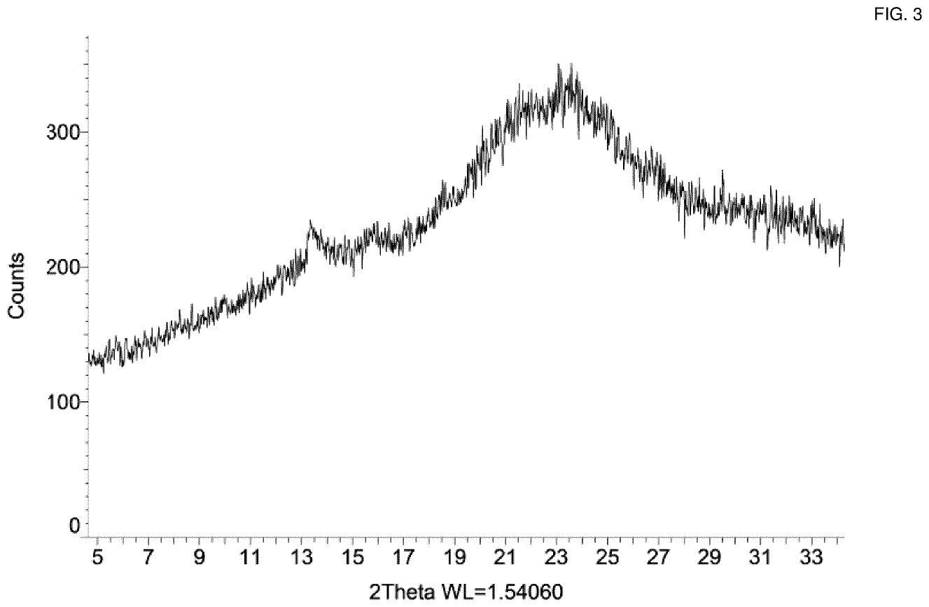

An illustrative PXRD diffractogram of upadacitinib Form APO-III, as prepared in Example 3, is shown in FIG. 3. In FIG. 3, the PXRD diffractogram of Form APO-III exhibits a broad halo lacking sharp peaks.

As described in Example 3, upadacitinib Form APO-III can be prepared by exposing upadacitinib Form APO-I to warm, moist air, preferably air having a relative humidity of from about 60% to about 100%, at a suitable temperature, preferably from about 35° C. to about 45° C., for a suitable time, to afford upadacitinib Form APO-III having a PXRD diffractogram consistent with FIG. 3.

In a fourth embodiment of the present disclosure, there is provided a new crystalline form of upadacitinib, upadacitinib Form APO-IV, comprising upadacitinib and orotic acid. In some embodiments, in upadacitinib Form APO-IV, the molar ratio of upadacitinib to orotic acid is about 1:2. In a further embodiment of Form APO-IV, the crystalline form is an orotic acid saltco-crystal wherein about 1 mole equivalent of orotic acid is co-crystallised and about 1 mole equivalent of orotic acid is ionized.

Upadacitinib Form APO-IV can be characterized by a PXRD diffractogram comprising, among other peaks, characteristic peaks, expressed in degrees 2θ (±0.2°), at 5.3°, 12.0°, and 17.7°. In some embodiments, the PXRD diffractogram further comprises at least three peaks, expressed in degrees 2θ (±0.2°), selected from the group consisting of 8.8°, 10.6°, 12.9°, 14.6°, 15.9°, and 16.7°. In some embodiments, the PXRD diffractogram further comprises peaks, expressed in degrees 2θ (±0.2°), at 8.8°, 10.6°, 12.9°, 14.6°, 15.9°, and 16.7°.

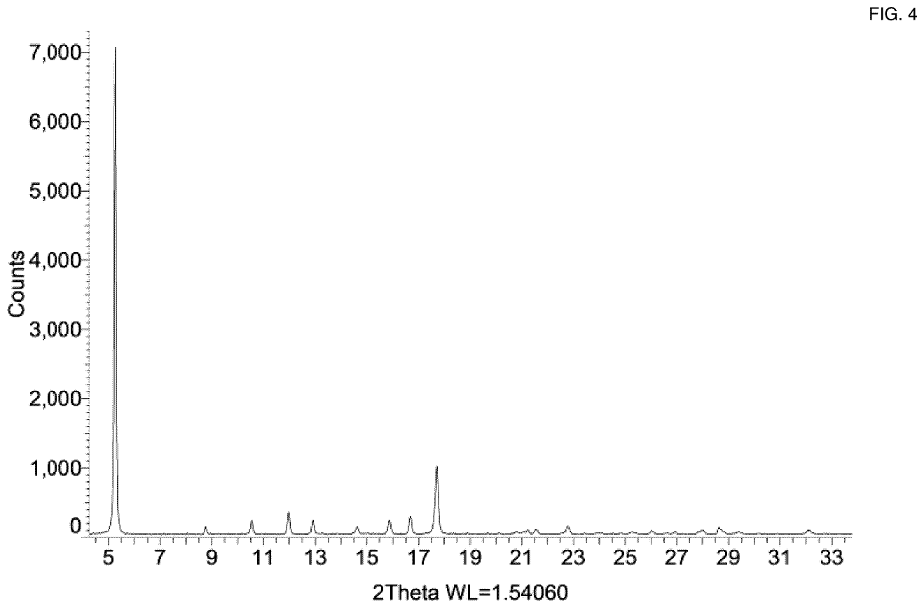

An illustrative PXRD diffractogram of upadacitinib Form APO-IV, as prepared in Example 4, is shown in FIG. 4. A peak listing, comprising representative peaks from the PXRD diffractogram in FIG. 4, and their relative intensities, is provided in Table 3. Although illustrative of the PXRD diffractogram that is provided for the upadacitinib Form APO-IV of the present disclosure, the relative intensities of the peaks are variable. Thus, depending on a particular sample, the prominence or relative intensity of the peaks observed may differ from those in the illustrative PXRD diffractogram and peak listing.

TABLE 3
Relative Peak Intensities of Upadacitinib
Form APO-IV from FIG. 4

| Angle (2θ) | Relative intensity (%) |
|---|---|
| 5.26 | 100.0 |
| 8.76 | 1.6 |
| 10.55 | 2.9 |
| 11.98 | 4.6 |
| 12.91 | 3.0 |
| 14.63 | 1.5 |
| 15.88 | 3.1 |
| 16.69 | 3.5 |
| 17.71 | 14.8 |
| 22.78 | 1.8 |
| 28.64 | 1.7 |

As described in Example 4, upadacitinib Form APO-IV can be prepared by combining upadacitinib and orotic acid, in some embodiments from about one to about two mole equivalents of orotic acid monohydrate, in a first suitable solvent, for example a C1-C3 alcohol, preferably methanol, and evaporating to dryness to afford a sample. Combining the sample with a second suitable solvent, for example a C1-C3 alcohol, preferably methanol, and applying methods, if necessary, such as sonication, stirring, and/or heat cycling between about 0° C. and about 60° C. for a suitable time and isolating the resulting solid and drying, if necessary, preferably in vacuo, affords upadacitinib Form APO-IV having a PXRD diffractogram consistent with FIG. 4.

In a fifth embodiment of the present disclosure, there is provided a new crystalline form of upadacitinib, upadacitinib Form APO-V, comprising upadacitinib and orotic acid. In some embodiments, in upadacitinib Form APO-V, the molar ratio of upadacitinib to orotic acid is about 1:2. In a further embodiment of Form APO-V, the crystalline form is an orotic acid saltco-crystal wherein about 1 mole equivalent of orotic acid is co-crystallised and about 1 mole equivalent of orotic acid is ionized.

Upadacitinib Form APO-V can be characterized by a PXRD diffractogram comprising, among other peaks, characteristic peaks, expressed in degrees 2θ (±0.2°), at 4.7°, 9.5°, and 14.2°. In some embodiments, the PXRD diffractogram further comprises at least three peaks, expressed in degrees 2θ (±0.2°), selected from the group consisting of 11.2°, 11.8°, 15.3°, 16.0°, 19.0°, and 20.5°. In some embodiments, the PXRD diffractogram further comprises peaks, expressed in degrees 2θ (±0.2°), at 11.2°, 11.8°, 15.3°, 16.0°, 19.0°, and 20.5°.

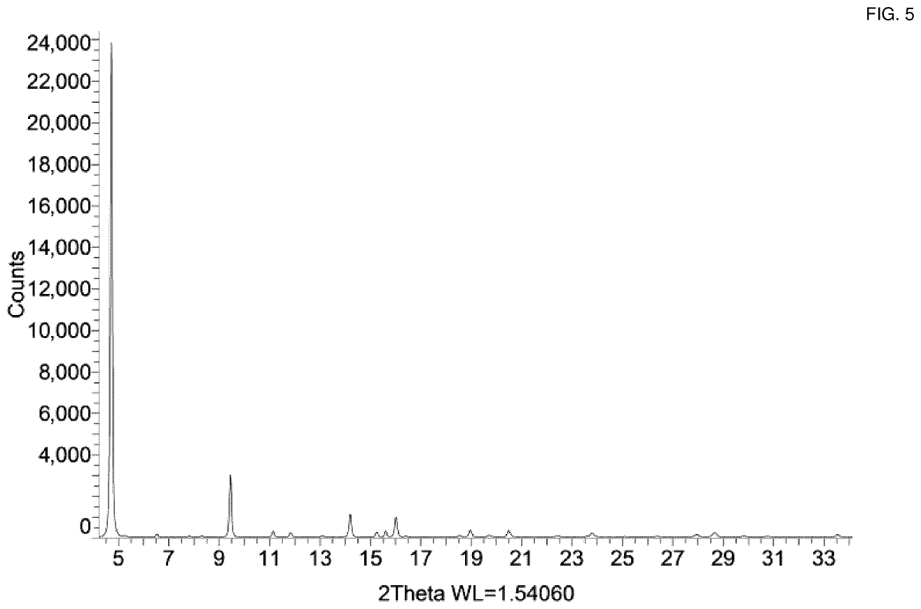

An illustrative PXRD diffractogram of upadacitinib Form APO-V, as prepared in Example 5, is shown in FIG. 5. A peak listing, comprising representative peaks from the PXRD diffractogram in FIG. 5, and their relative intensities, is provided in Table 4. Although illustrative of the PXRD diffractogram that is provided for the upadacitinib Form APO-V of the present disclosure, the relative intensities of the peaks are variable. Thus, depending on a particular sample, the prominence or relative intensity of the peaks observed may differ from those in the illustrative PXRD diffractogram and peak listing.

TABLE 4
Relative Peak intensities of Upadacitinib
Form APO-V from FIG. 5

| Angle (2θ) | Relative intensity (%) |
|---|---|
| 4.72 | 100.0 |
| 9.45 | 13.0 |
| 11.15 | 1.2 |
| 11.83 | 0.9 |
| 14.19 | 5.0 |
| 15.25 | 1.1 |
| 15.60 | 1.2 |
| 16.02 | 4.3 |
| 18.97 | 1.6 |
| 20.48 | 1.5 |
| 23.79 | 0.9 |
| 28.66 | 1.0 |

As described in Example 5, upadacitinib Form APO-V can be prepared by combining upadacitinib and orotic acid, in some embodiments from about one to about two mole equivalents of orotic acid monohydrate, in a first suitable solvent, for example a C1-C3 alcohol, preferably trifluoroethanol, and evaporating to dryness to afford a first sample. Treating the first sample with minimal water and applying methods, if necessary, such as sonication, stirring, and/or heat cycling between about 0° C. and about 60° C. for a suitable time affords a second sample. Triturating the second sample with a second suitable solvent, preferably methanol, and isolating the resulting solid affords upadacitinib Form APO-V having a PXRD diffractogram consistent with FIG. 5. Upadacitinib Form APO-V undergoes conversion to upadacitinib Form APO-IV upon drying, particularly in vacuo.

In a sixth embodiment of the present disclosure, there is provided a new crystalline form of upadacitinib, upadacitinib Form APO-VI, comprising upadacitinib and magnesium orotate. In an embodiment, upadacitinib Form APO-VI, is a magnesium orotate co-crystal.

Upadacitinib Form APO-VI can be characterized by a PXRD diffractogram comprising, among other peaks, characteristic peaks, expressed in degrees 2θ (±0.2°), at 8.1°, 10.6° and 28.6°. In some embodiments, the PXRD diffractogram further comprises at least three peaks, expressed in degrees 2θ (±0.2°), selected from the group consisting of 14.6°, 16.3°, 17.7°, 21.4°, 22.2°, and 26.0°. In some embodiments, the PXRD diffractogram further comprises peaks, expressed in degrees 2θ (±0.2°), at 14.6°, 16.3°, 17.7°, 21.4°, 22.2°, and 26.0°.

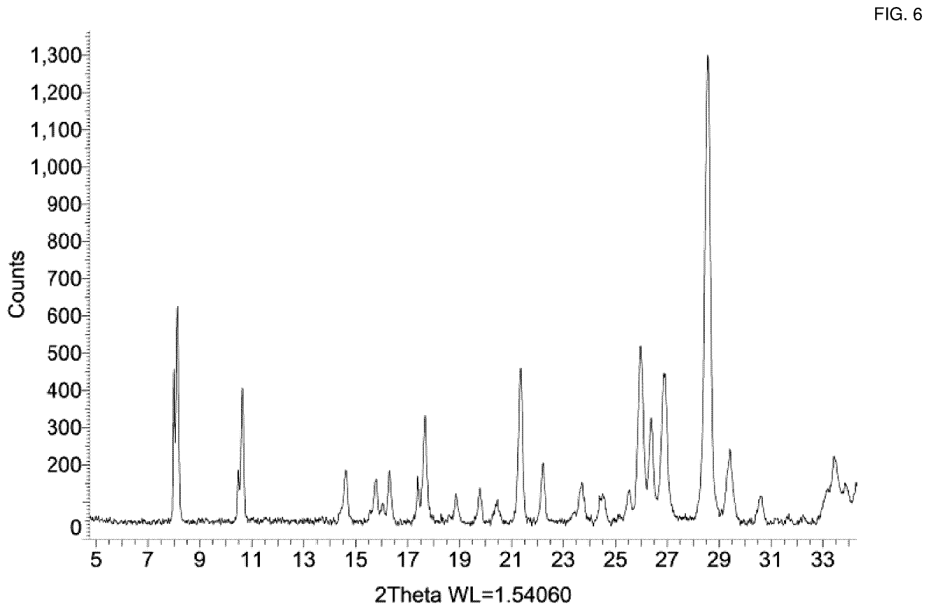

An illustrative PXRD diffractogram of upadacitinib Form APO-VI, as prepared in Example 6, is shown in FIG. 6. A peak listing, comprising representative peaks from the PXRD diffractogram in FIG. 6, and their relative intensities, is provided in Table 5. Although illustrative of the PXRD diffractogram that is provided for the upadacitinib Form APO-VI of the present disclosure, the relative intensities of the peaks are variable. Thus, depending on a particular sample, the prominence or relative intensity of the peaks observed may differ from those in the illustrative PXRD diffractogram and peak listing.

TABLE 5
Relative Peak Intensities of Upadacitinib
Form APO-VI from FIG. 6

| Angle (2θ) | Relative intensity (%) |
|---|---|
| 8.00 | 33.1 |
| 8.14 | 44.1 |
| 10.48 | 11.6 |
| 10.64 | 27.9 |
| 14.62 | 10.8 |
| 15.79 | 8.4 |
| 16.30 | 11.9 |
| 17.40 | 11.3 |
| 17.68 | 22.4 |
| 18.86 | 6.5 |
| 19.79 | 7.2 |
| 21.36 | 31.4 |
| 22.22 | 12.3 |
| 25.98 | 37.6 |
| 26.38 | 22.7 |
| 26.90 | 29.3 |
| 28.57 | 100.0 |
| 29.42 | 16.0 |

As described in Example 6, upadacitinib Form APO-VI can be prepared by combining upadacitinib with about equimolar amounts of magnesium orotate in a suitable solvent, for example an aqueous C1-C3 alcohol mixture, preferably aqueous methanol, and evaporating to dryness to afford a sample. Treating the sample with a suitable solvent, preferably 2-propanol, and applying methods, if necessary, such as sonication, stirring, and/or heat cycling between about 0° C. and about 60° C. for a suitable time and isolating the resulting solid affords upadacitinib Form APO-VI having a PXRD diffractogram consistent with FIG. 6.

In a seventh embodiment of the present disclosure, there is provided a new crystalline form of upadacitinib, upadacitinib Form APO-VII, comprising upadacitinib and magnesium fumarate. In an embodiment, upadacitinib Form APO-VII is a magnesium fumarate co-crystal.

Upadacitinib Form APO-VII can be characterized by a PXRD diffractogram comprising, among other peaks, characteristic peaks, expressed in degrees 2θ (±0.2°), at 9.1°, 11.8°, and 26.6°. In some embodiments, the PXRD diffractogram further comprises at least three peaks, expressed in degrees 2θ (±0.2°), selected from the group consisting of 12.4°, 16.1°, 17.1°, 18.3°, 21.7°, and 22.6°. In some embodiments, the PXRD diffractogram further comprises peaks, expressed in degrees 2θ (±0.2°), at 12.4°, 16.1°, 17.1°, 18.3°, 21.7°, and 22.6°.

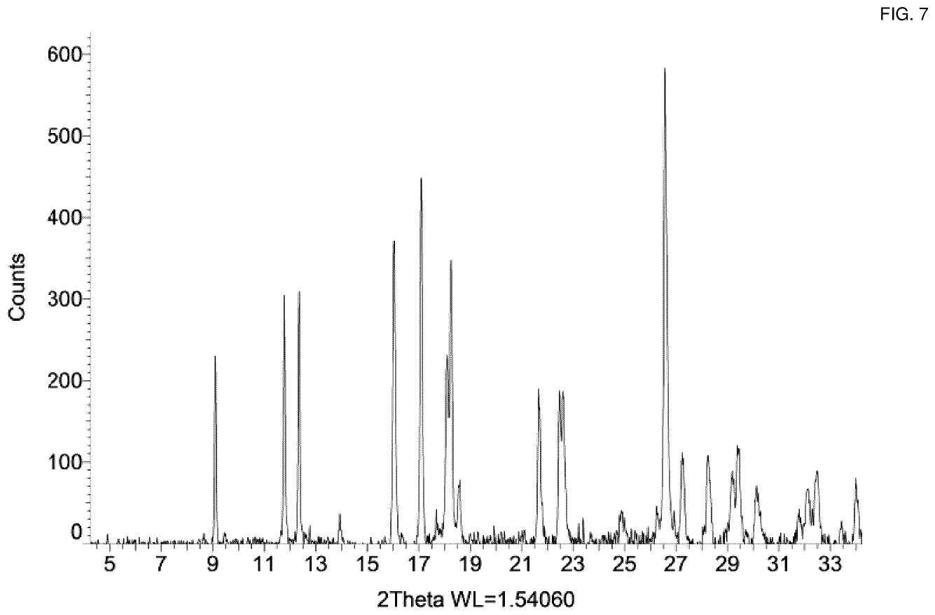

An illustrative PXRD diffractogram of upadacitinib Form APO-VII, as prepared in Example 7, is shown in FIG. 7. A peak listing, comprising representative peaks from the PXRD diffractogram in FIG. 7, and their relative intensities, is provided in Table 6. Although illustrative of the PXRD diffractogram that is provided for the upadacitinib Form APO-VII of the present disclosure, the relative intensities of the peaks are variable. Thus, depending on a particular sample, the prominence or relative intensity of the peaks observed may differ from those in the illustrative PXRD diffractogram and peak listing.

TABLE 6
Relative Peak Intensities of Upadacitinib
Form APO-VII from FIG. 7

| Angle (2θ) | Relative intensity (%) |
|---|---|
| 9.11 | 34.4 |
| 11.79 | 46.4 |
| 12.36 | 45.6 |
| 16.05 | 52.9 |
| 17.10 | 68.6 |
| 18.12 | 31.8 |
| 18.27 | 51.7 |
| 18.60 | 10.1 |
| 21.67 | 33.6 |
| 22.47 | 33.1 |
| 22.61 | 31.8 |
| 26.58 | 100.0 |
| 27.24 | 18.3 |
| 28.23 | 22.1 |
| 29.19 | 13.5 |
| 29.45 | 13.5 |
| 30.14 | 10.4 |
| 32.11 | 11.6 |

As described in Example 7, upadacitinib Form APO-VII can be prepared by combining upadacitinib with about equimolar amounts of magnesium fumarate in a suitable solvent, preferably an aqueous C1-C3 alcohol mixture, most preferably aqueous methanol, and evaporating to dryness to afford a sample. Treating the sample with a suitable solvent, preferably methyl ethyl ketone, and applying methods, if necessary, such as sonication, stirring, and/or heat cycling between about 0° C. and about 60° C. for a suitable time and isolating the resulting solid affords upadacitinib Form APO-VII having a PXRD diffractogram consistent with FIG. 7.

In an eighth embodiment of the present disclosure, there is provided a new crystalline form of upadacitinib, upadacitinib Form APO-VIII, comprising upadacitinib and magnesium citrate. In an embodiment, upadacitinib Form APO-VIII, is a magnesium citrate co-crystal.

Upadacitinib Form APO-VIII can be characterized by a PXRD diffractogram comprising, among other peaks, characteristic peaks, expressed in degrees 2θ (±0.2°), at 9.0°, 11.5°, and 16.2°.

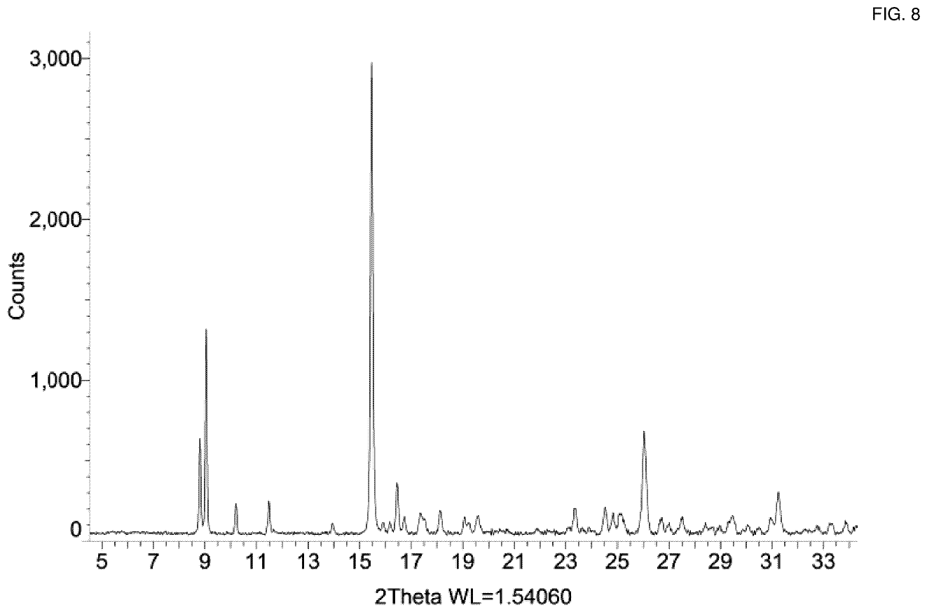

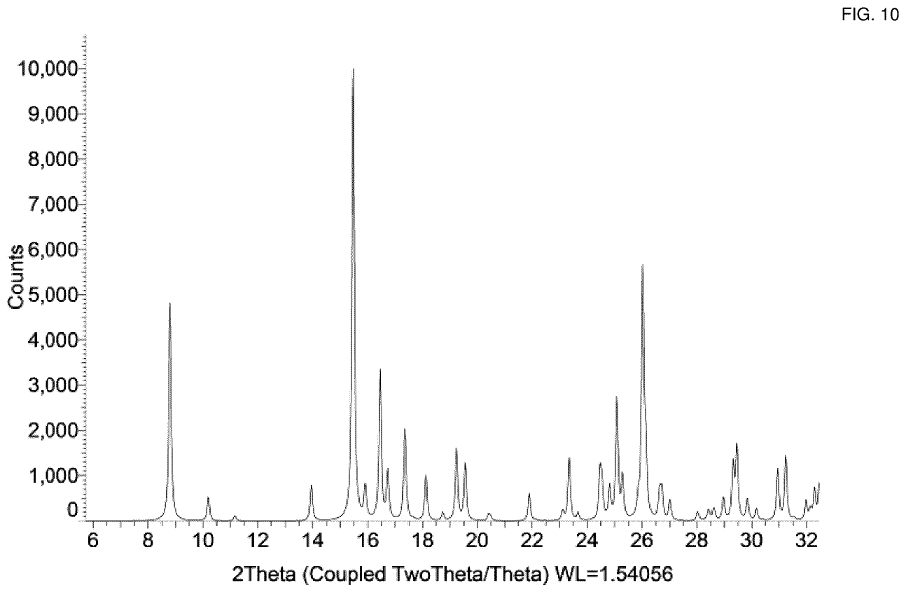

An illustrative PXRD diffractogram of a mixture of upadacitinib Form APO-VIII and trimagnesium dicitrate polyhydrate, as prepared in Example 8, is shown in FIG. 8. A peak listing, comprising representative peaks of Form APO-VIII from the PXRD diffractogram in FIG. 8, and their relative intensities, is provided in Table 7. Although illustrative of the PXRD diffractogram that is provided for the upadacitinib Form APO-VIII of the present disclosure, the relative intensities of the peaks are variable. Thus, depending on a particular sample, the prominence or relative intensity of the peaks observed may differ from those in the illustrative PXRD diffractogram and peak listing. A peak listing, and their relative intensities, from the PXRD diffractogram of trimagnesium dicitrate polyhydrate in FIG. 10 is provided in Table 8 for reference.

TABLE 7
Relative Peak Intensities of Upadacitinib
Form APO-VIII from FIG. 8

| Angle (2θ) | Relative intensity (%) |
|---|---|
| 9.05 | 43.3 |
| 11.49 | 6.9 |
| 16.18 | 2.7 |

TABLE 8
Relative Peak Intensities of Trimagnesium
Dicitrate Polyhydrate from FIG. 10

| Angle (2θ) | Relative intensity (%) |
|---|---|
| 8.80 | 48.7 |
| 10.20 | 5.3 |
| 13.95 | 7.8 |
| 15.48 | 100.0 |
| 15.91 | 7.9 |
| 16.46 | 33.4 |
| 16.74 | 11.1 |
| 17.36 | 20.1 |
| 18.13 | 9.7 |
| 19.24 | 16.0 |
| 19.56 | 12.8 |
| 21.90 | 6.1 |
| 23.34 | 13.9 |
| 24.48 | 12.8 |
| 24.82 | 8.1 |
| 25.08 | 27.4 |
| 25.28 | 10.5 |
| 26.03 | 56.7 |
| 26.71 | 8.1 |
| 27.02 | 4.5 |
| 29.46 | 16.9 |
| 30.95 | 11.3 |
| 31.24 | 14.3 |

As described in Example 8, upadacitinib Form APO-VIII can be prepared by combining upadacitinib with magnesium citrate, in some embodiments about equimolar amounts of magnesium citrate pentahydrate, in a suitable solvent, for example an aqueous C1-C3 alcohol mixture, preferably aqueous methanol, and evaporating to dryness to afford a sample. Treating the sample with a suitable solvent, preferably 2-propanol, and applying methods, if necessary, such as sonication, stirring, and/or heat cycling between about 0° C. and about 60° C. for a suitable time and isolating the resulting solid affords a mixture of upadacitinib Form APO-VIII and trimagnesium dicitrate polyhydrate having a PXRD diffractogram consistent with FIG. 8.

In a further embodiment of the disclosure, there is provided a pharmaceutical composition comprising a crystalline form of upadacitinib comprising upadacitinib and a magnesium salt, with one or more pharmaceutically acceptable excipients. In another embodiment of the disclosure, there is provided a pharmaceutical composition comprising a crystalline form of upadacitinib comprising upadacitinib and a magnesium salt selected from the group consisting of magnesium chloride, magnesium acetate, magnesium orotate, magnesium fumarate, and magnesium citrate. In another embodiment of the disclosure, there is provided a pharmaceutical composition comprising an amorphous solid dispersion comprising upadacitinib and magnesium chloride. In another embodiment of the disclosure, there is provided a pharmaceutical composition comprising a crystalline form of upadacitinib comprising upadacitinib and orotic acid. In another embodiment of the disclosure, there is provided a pharmaceutical composition comprising a solid form of upadacitinib selected from the group consisting of upadacitinib Form APO-I, Form APO-II, Form APO-III, Form APO-IV, Form APO-V, Form APO-VI, Form APO-VII, Form APO-VIII and mixtures thereof. The amount of solid form(s) present in the composition, expressed as the equivalent anhydrous upadacitinib free base, can range from about 1% w/w to about 5% w/w, the remainder comprising pharmaceutically acceptable excipients comprising, for example, from about 30% w/w to about 60% w/w diluent, from about 10% w/w to about 35% w/w pH modifier such as tartaric acid, and from about 15% w/w to about 25% w/w release control polymer. In some embodiments, the pharmaceutical composition is a solid dosage form suitable for oral administration, such as a capsule, tablet, pill, powder, or granulate. In some embodiments, the pharmaceutical composition is a tablet, preferably an extended-release tablet. In some embodiments, the pharmaceutical composition provides a dose of the upadacitinib solid form that is equivalent to the 15 mg of upadacitinib found in RINVOQ© drug products.

Suitable pharmaceutically acceptable excipients are preferably inert with respect to the crystalline form of upadacitinib of the present disclosure, and may comprise, for example, one or more excipients selected from binders such as lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatine, polyvinylpyrrolidone (PVP) and/or sodium alginate; fillers and/or diluents such as lactose, sugar, starches, modified starches, mannitol, sorbitol, inorganic salts, cellulose derivatives (e.g., microcrystalline cellulose, cellulose), calcium sulphate, xylitol and/or lactitol; disintegrants such as croscarmellose sodium, crospovidone, polyvinylpyrrolidone, sodium starch glycollate, corn starch, microcrystalline cellulose, hydroxypropyl methylcellulose and/or hydroxypropyl cellulose; lubricants such as magnesium stearate, magnesium lauryl stearate, sodium stearyl fumarate, stearic acid, calcium stearate, zinc stearate, potassium benzoate, sodium benzoate, myristic acid, palmitic acid, mineral oil, hydrogenated castor oil, medium-chain triglycerides, poloxamer, polyethylene glycol and/or talc; and/or dispersants and/or solubility enhancing agents, such cyclodextrins, glyceryl monostearate, hypromellose, meglumine, poloxamer, polyoxyethylene castor oil derivatives, polyoxyethylene stearates, polyoxylglycerides, povidone, and/or stearic acid. Other excipients comprising preservatives, stabilisers, anti-oxidants, silica flow conditioners, anti-adherents, pH modifiers and/or glidants may be added as required. Other suitable excipients and the preparation of solid oral dosage forms is well known to person of skill in the art, and is described generally, for example, in Remington The Science and Practice of Pharmacy 21^{st} Edition (Lippincott Williams & Wilkins: Philadelphia; 2006; Chapter 45).

Optionally, when the pharmaceutical compositions are solid dosage forms, the solid dosage forms may be prepared with coatings, such as enteric coatings and extended-release coatings, using standard pharmaceutical coatings. Such coatings, and their application, are well known to persons skilled in the art, and are described, for example, in Remington The Science and Practice of Pharmacy 21^{st} Edition (Lippincott Williams & Wilkins: Philadelphia; 2006; Chapter 46).

In some embodiments, there is provided a method of treating a Janus kinase (JAK)-associated condition, such as rheumatoid arthritis, psoriatic arthritis, atopic dermatitis, ulcerative colitis, or ankylosing spondylitis, comprising administering to a human subject in need thereof a therapeutically effective amount of crystalline form according to the first, second, third, fourth, fifth or sixth embodiments, or the amorphous solid dispersion of the seventh embodiment of the disclosure, or a combination thereof, or the pharmaceutical composition of the ninth embodiment of the disclosure.

Also provided herein are methods for treating a Janus kinase (JAK)-associated condition, such as rheumatoid arthritis, psoriatic arthritis, atopic dermatitis, ulcerative colitis, or ankylosing spondylitis, comprising administering to a human subject in need thereof a therapeutically effective amount of a crystalline form disclosed herein or a combination of crystalline forms, or the amorphous solid dispersion disclosed herein, or a pharmaceutical composition disclosed herein, to a human subject in a therapeutically effective amount for the treatment of a Janus kinase (JAK)-associated condition. As used herein, the phrase “therapeutically effective amount” means that amount of upadacitinib that will elicit a biological or medical response of a tissue, system, or patient that is being sought by the administrator (such as a researcher, doctor, or veterinarian) which includes alleviation of the symptoms of the condition or disease being treated and the prevention, slowing, or halting of progression of the condition or disease, including but not limited to a Janus kinase (JAK)-associated condition, for example a dose of upadacitinib solid form that is equivalent to the 15 mg of upadacitinib (anhydrous basis) found in RINVOQ® drug products.

### EXAMPLES

The following non-limiting examples are illustrative of some of the embodiments of the disclosure described herein.

The upadacitinib used as a starting material in the following examples was in the amorphous form, except where otherwise indicated. The PXRD diffractogram of trimagnesium dicitrate polyhydrate shown in FIG. 10 was generated from single crystal X-ray diffraction (SCXRD) data reported in Kaduk, J. A, Acta Crystallographica Section E: Crystallographic Communications, 2020, 76, 1611.

### PXRD Analysis:

PXRD diffractograms were recorded on a Bruker D8 Discover powder X-ray diffractometer (Bruker AXS LLC, Karlsruhe, Germany). The generator was a Incoatec Microfocus Source (IpS) Cu tube (A=1.5406 Å) with a voltage of 50 kV and current of 1.00 mA, using a divergence slit of 0.1 mm and collimator of 2.0 mm. For each sample, two frames were collected using a still scan with a PILATUS3 R 100K-A detector at the distance of 294.9 mm from the sample. Raw data were evaluated using the program DIFFRAC.EVA (Bruker AXS LLC, Karlsruhe, Germany).

### Analysis Method for Determining the Chromatographic Purity of Upadacitinib Form APO-I

The method shown in Table 9 was used to determine the chromatographic purity of samples of upadacitinib Form APO-I as provided in examples 10 and 11.

TABLE 9
HPLC method for the determination of chromatographic
purity of upadacitinib Form APO-I

| Instrument | Waters ® 2695 | | |
|---|---|---|---|
| Column | Atlantis ™ dC18, 4.6 × 250 mm, 5 μm | | |
| Column Temp. | 30° C. | | |
| Sample temp. | 25° C. | | |
| Mobile phase | Mobile Phase A: 0.77 g HPLC grade | | |
| | ammonium acetate in 1 L deionized water | | |
| | Mobile Phase B: HPLC grade acetonitrile | | |
| Gradient Program | Time (min) | % Solution A | % Solution B |
| | 0 | 70 | 30 |
| | 20 | 35 | 65 |
| | 21 | 10 | 90 |
| | 26 | 10 | 90 |
| | 27 | 70 | 30 |
| | 35 | 70 | 30 |

| | |
|---|---|
| Flow rate | 1.0 mL/minute |
| Injection volume | 5 μL |
| Detector | Waters ® W2996 PDA detector, set to |
| Run time | 230 nm detection 35 minutes |
| Sample prep. | Weighed accurately between 5 mg and 10 mg |
| | into a 4 dram vial and dissolved in 1000 |
| | volumes of HPLC grade methanol |
| Retention times | Upadacitinib: 8.69 min (RRT 1.00) |
| | UPA-III*: 9.33 min (RRT 1.07) |

*UPA-III is (3S,4S)-3-ethyl-4-(3H-imidazo[1,2-a]pyrrolo[2,3-e]pyrazin-8-yl)-N-(2,2,2-trifluoroethyl)pyrrolidine-1-carboxamide)

### Example 1: Preparation of Upadacitinib Form APO-I

Upadacitinib (333.7 mg) and anhydrous magnesium chloride (42.5 mg) were combined in a vial with methanol (3 mL). The resulting solution was allowed to evaporate overnight with the assistance of a stream of nitrogen. Ethyl acetate (3 mL) was added to the resulting yellow oil, and the sample was perturbed with a spatula, sonicated three times (15 minutes each time), and subjected to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1 h. The resulting very thick/viscous suspension was vacuum filtered, and the isolated solid was washed twice with ethyl acetate (3 mL) before drying in vacuo (ca. 1 Torr) overnight to afford upadacitinib Form APO-I (387.5 mg) as a white powder. The PXRD diffractogram of the sample is shown in FIG. 1. Peaks attributable to crystalline magnesium chloride (including hydrates/solvates) were not observed in the PXRD diffractogram. ¹H NMR analysis of the sample revealed differences when compared to upadacitinib (amorphous), presumably due to the presence of magnesium chloride. Karl Fischer (KF) analysis of the sample showed a water content of 8.5 wt %, consistent with ca. 2.25 mole equivalents of water. The PXRD of a sample of Form APO-I was substantially unchanged (disregarding any change of relative intensities of peaks) following storage in a capped vial at 40° C./75% RH (relative humidity) for 15 days.

¹H-NMR of upadacitinib Form APO-I (DMSO-d₆, 300 MHz) δ: 12.32 (s, 1H), 8.56 (s, 1H), 7.46 (s, 1H), 7.45 (t, J=3.1 Hz, 1H), 7.01 (m, 2H), 4.35 (m, 1H), 3.84 (m, 4H), 3.68 (d of d, J=6.9, 10.2 Hz, 1H), 3.27 (d of d, J=6.1, 10.0 Hz, 1H), 2.56 (m, 1H), 1.10 (m, 1H), 0.80 (m, 1H), 0.64 (t, J=7.3 Hz, 3H).

### Example 2: Preparation of Form APO-II

A portion (0.75 mL) of a solution containing upadacitinib (332.6 mg) and magnesium acetate tetrahydrate (185.4 mg) in a 1:1 mixture of methanol:trifluoroethanol (3 mL) was allowed to evaporate overnight with the assistance of a stream of nitrogen. To the sample was added ethyl acetate (0.4 mL), and the resulting suspension was subjected to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1 h. Vacuum filtration afforded a grey powder, which was washed with ethyl acetate (2×0.5 mL) to afford a white powder. Drying the powder in vacuo (ca. 1 Torr) for 4 hours afforded upadacitinib Form APO-II as a white powder (31.6 mg) having a PXRD diffractogram as shown in FIG. 2. ¹H NMR analysis sample indicated a molar ratio of upadacitinib: magnesium acetate of about 1:5. Peaks attributable to crystalline magnesium acetate, including hydrates thereof (as disclosed in Scheurell, K. et. al. Z. Anorg. Allg. Chem. 2012, 1265-1273; Miller, J. R. et. al. J. Solid State Chem. 2019, 270, 1-10) were not detected in the PXRD diffractogram shown in FIG. 2. The PXRD of a sample of the powder was substantially unchanged (i.e. disregarding relative intensities of peaks) following storage in a capped vial at 40° C./75% RH (relative humidity) for 5 days.

¹H-NMR of upadacitinib Form APO-II (DMSO-d₆, 300 MHz) δ: 12.31 (s, 1H), 8.57 (s, 1H), 7.46 (s, 1H), 7.44 (d, J=3.4 Hz, 1H), 6.98 (m, 2H), 4.35 (q, J=5.8 Hz, 1H), 3.84 (m, 4H), 3.68 (d of d, J=7.0, 10.2 Hz, 1H), 3.26 (d of d, J=6.1, 10.2 Hz, 1H), 2.56 (m, 1H), 1.10 (m, 1H), 0.80 (m, 1H), 0.63 (t, J=7.3 Hz, 3H).

### Example 3: Preparation of Upadacitinib Form APO-III

A sample of upadacitinib Form APO-II (31.0 mg) was placed in a test tube and left uncovered at 40° C./75% RH for 5 days. The resulting white powder was placed in vacuo (ca. 1 Torr) for one hour to afford upadacitinib Form APO-III (30.8 mg). The PXRD diffractogram of the sample is shown in FIG. 3. The PXRD diffractogram of a sample was unchanged after being subjected to vacuum for 5 days at room temperature.

### Example 4: Preparation of Upadacitinib Form APO-IV

Upadacitinib (59.4 mg) was dissolved in methanol (1 mL) and added to orotic acid monohydrate (22.4 mg). The mixture was sonicated twice for 15 minutes each to afford a white suspension, and the solvent was allowed to evaporate overnight (with the assistance of a nitrogen flow) to afford a white powder. PXRD analysis of this crude sample (66.0 mg) indicated a mixture of upadacitinib Form APO-IV and upadacitinib Form APO-V. A portion (36.7 mg) of the mixture was dissolved in methanol (0.3 mL) and subjected to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1h. A white powder was isolated by vacuum filtration, washed with methanol (0.5 mL), and dried in vacuo (ca. 1 Torr) to afford upadacitinib Form APO-IV (16.8 mg) as a white powder. The PXRD diffractogram of the sample is shown in FIG. 4. ¹H NMR analysis of the sample indicated a molar ratio of upadacitinib: orotic acid of about 1:2. The sample exhibited conversion to upadacitinib Form APO-V under ambient conditions, which was accelerated by higher humidities and temperatures.

¹H-NMR of upadacitinib Form APO-IV (300 MHz, DMSO-d₆): 12.34 (s, 1H), 11.32 (s, 2H), 10.86 (s, 2H), 8.60 (s, 1H), 7.51 (s, 1H), 7.48 (t, J=3.1 Hz, 1H), 7.02 (d of d, J=1.8, 3.2 Hz, 1H), 6.97 (t, J=6.3 Hz, 1H), 5.99 (s, 2H), 4.36 (q, J=6.2 Hz, 1H), 3.82 (m, 4H), 3.69 (d of d, J=7.1, 10.3 Hz, 1H), 3.27 (d of d, J=6.1, 10.2 Hz, 1H), 2.56 (m, 1H), 1.10 (m, 1H), 0.82 (m, 1H), 0.64 (t, J=7.3 Hz, 3H).

### Example 5: Preparation of Upadacitinib Form APO-V

Upadacitinib (55.2 mg) and orotic acid monohydrate (24.3 mg) were combined in trifluoroethanol (1.5 mL). The mixture was sonicated for 1 minute and the resulting suspension was allowed to evaporate overnight under a stream of nitrogen. Water (0.15 mL) was added and the mixture was subjected to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1h. Following this, the clear colourless mother liquor was decanted to isolate a residue, which was dried in vacuo (ca. 1 Torr) overnight to afford a crude sample (57.3 mg). A portion (14.0 mg) of this crude sample was suspended in methanol (0.45 mL) to afford a white suspension, which was collected by filtration to afford upadacitinib Form APO-V as a white powder. The PXRD diffractogram of the sample is shown in FIG. 5. The sample was prone to conversion to Form APO-IV upon drying.

### Example 6: Preparation of Upadacitinib Form APO-VI

An aliquot (190 μL) of a magnesium orotate suspension (prepared by combining magnesium hydroxide (8.5 mg) and orotic acid (50.1 mg) in water (1.8 mL)) was combined with a solution (40 μL) of upadacitinib (571.7 mg) in methanol (4.6 mL), and the mixture was allowed to evaporate under a nitrogen stream overnight, and further dried in vacuo (ca.1 Torr) for 3 hours. 2-propanol (40 μL) was added and the reaction mixture was subjected to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1 h. The mother liquor was removed to afford upadacitinib Form APO-VI. The PXRD diffractogram of the sample is shown in FIG. 6.

### Example 7: Preparation of Upadacitinib Form APO-VII

An aliquot (160 μL) of a magnesium fumarate (1:1) suspension (prepared by combining magnesium hydroxide (8.5 mg) and fumaric acid (16.4 mg) in water (1.8 mL)) was combined with a solution (40 μL) of upadacitinib (571.7 mg) in methanol (4.6 mL), and the mixture was allowed to evaporate under a nitrogen stream overnight, and further dried in vacuo (ca.1 Torr) for 3 hours. Methyl ethyl ketone (50 μL) was added and the mixture was subjected to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1 h. The mother liquor was removed to afford upadacitinib Form APO-VII. The PXRD diffractogram of the sample is shown in FIG. 7.

### Example 8: Preparation of a Mixture of Upadacitinib Form APO-VIII and Trimagnesium DiCitrate Polyhydrate

A solution (40 μL) of upadacitinib (571.7 mg) in methanol (4.6 mL) was combined with a suspension (160 μL) of magnesium citrate pentahydrate (31.2 mg) in water (1.76 mL), and the mixture was allowed to evaporate with the assistance of nitrogen stream overnight, and further dried in vacuo (ca. 1 Torr) for 3 hours. 2-propanol (40 μL) was added and the mixture was subjected to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1 h. The mother liquor was removed to afford a mixture of upadacitinib Form APO-VIII and crystalline trimagnesium dicitrate polyhydrate. The PXRD diffractogram of the sample is shown in FIG. 8. Comparison of the PXRD diffractogram of FIG. 8 with the PXRD diffractogram of trimagnesium dicitrate polyhydrate shown in FIG. 10, confirmed presence of the latter salt in the sample.

### Example 9: Preparation of Upadacitinib Form APO-I

A solution of anhydrous magnesium chloride (9.6 mg) in methanol (0.25 mL) was added to upadacitinib (77.2 mg), and the mixture was sonicated until complete dissolution. A single drop of water was added, which afforded a small amount of white precipitate which re-dissolved within seconds. Ethyl acetate (1 mL) was added, affording a small amount of white precipitate which re-dissolved within seconds. The solvent was reduced in vacuo (ca. 105 Torr, 30° C.) to 0.5 mL, at which point an additional amount of ethyl acetate (0.75 mL) was added and white precipitation was observed. The solvent was again reduced in vacuo (90-105 Torr, 30° C.) to 0.5 mL and the mixture was stirred for 5 hours at 50° C. and allowed to sit overnight at room temperature. Vacuum filtration followed by washing with ethyl acetate (1 mL) and drying overnight in vacuo (ca. 1 Torr) afforded upadacitinib Form APO-I as a white powder (64.1 mg) having suboptimal crystallinity by PXRD. The crystallinity of the solid was improved by suspending the solid in ethyl acetate (0.6 mL) and subjecting the suspension to the following temperature regime with stirring: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1 h. The resulting thick/viscous suspension was vacuum filtered, and the isolated solid was washed with ethyl acetate (2×1 mL) before drying in vacuo (ca. 1 Torr) for 3.5 hours to afford upadacitinib Form APO-I as a white powder (51.7 mg). The PXRD diffractogram of the powder was consistent with FIG. 1.

### Example 10: Preparation of Upadacitinib Form APO-I

A solution of crude upadacitinib (HPLC purity 98.71 a %, UPA-III content 1.18 a %) in 2-methyl tetrahydrofuran (about 3 mL), obtained from aqueous work-up of a preparative reaction mixture, was added to a solution of magnesium chloride (124.8 mg) in aqueous ethanol (0.35 mL water/2 mL ethanol) at 55° C. with stirring. To this solution was added ethyl acetate (3 mL). Within 10 minutes, upadacitinib Form APO-I seed crystals (40 mg) were added. Ethyl acetate was added portion-wise in 3 mL aliquots to the resulting suspension after 1 hour, 2 hours, and 3 hours. After stirring at 55° C. for one hour, the suspension was slowly cooled to 0° C. and stirred for another hour. A white solid was then isolated by vacuum filtration, and the filter cake was washed with acetone (2×5 mL). Drying overnight in vacuo (ca. 5 Torr) at 60° C. afforded upadacitinib Form APO-I (0.897 g) as a white solid. Karl Fischer (KF) analysis of the sample showed a water content of 7.9 wt %, consistent with ca. 2 mole equivalents of water. The solid contained about 0.24 wt % acetone by ¹H NMR analysis and an HPLC purity of 99.84 a % with UPA-III content of 0.16 a %.

### Example 11: Purification of Upadacitinib Form APO-I

A sample (2.603 g) of upadacitinib Form APO-I (HPLC purity 99.71 a %, UPA-Ill content 0.25 a %) was dissolved in aqueous ethanol (0.625 mL water/5.2 mL ethanol), followed by slow addition of ethyl acetate (54.6 mL). Seed crystals of upadacitinib Form APO-I (10 mg) were added after about 8 mL of ethyl acetate was added. The resulting suspension was filtered, washed with acetone (2×10 mL) and dried in vacuo (ca. 5 Torr) at about 55° C. overnight to afford purified upadacitinib Form APO-I (2.123 g) having an HPLC purity of 99.96 a % and UPA-III content 0.04 a %. The PXRD diffractogram of a sample prepared by this method is shown in FIG. 9. The PXRD of a sample of the material was substantially unchanged following storage in an uncapped vial at room temperature and ambient humidity (ca. 15-20% RH) for 37 days.

What is claimed is:

1. A crystalline form of upadacitinib comprising upadacitinib and a magnesium salt.

2. The crystalline form of claim 1, wherein the magnesium salt is selected form the group consisting of magnesium chloride, magnesium acetate, magnesium orotate, magnesium fumarate, and magnesium citrate.

3. The crystalline form of claim 2, wherein the molar ratio of upadacitinib to the magnesium salt is from about 1:0.4 to about 1:5.

4. The crystalline form of claim 3, wherein the crystalline form is a hydrate.

5. The crystalline form of claim 3, wherein the crystalline form is anhydrous.

6. A crystalline form of upadacitinib comprising upadacitinib and magnesium chloride.

7. The crystalline form of claim 6, wherein the molar ratio of upadacitinib to the magnesium chloride is about 1:0.4 to about 1:1.

8. The crystalline form of claim 7, wherein the crystalline form is a hydrate.

9. The crystalline form of upadacitinib of claim 7, characterized by a PXRD diffractogram comprising peaks, expressed in degrees 2θ (±0.2°), at 4.3°, 5.5°, and 21.1°.

10. The crystalline form of claim 9, further comprising at least three peaks, expressed in degrees 2θ (±0.2°), selected from the group consisting of: 8.1°, 8.7°, 13.3°, 15.7°, 17.1°, and 23.6°.

11. The crystalline form of claim 9, further comprising peaks, expressed in degrees 2θ (±0.2°), at 8.1°, 8.7°, 13.3°, 15.7°, 17.1°, and 23.6°.

12. The crystalline form of claim 7, providing a PXRD diffractogram comprising peaks in substantially the same positions (±0.2°2θ) as those shown in FIG. 1.

13. The crystalline form of claim 7, providing a PXRD diffractogram comprising peaks in substantially the same positions (±0.2°2θ) as those shown in FIG. 9.

14. An amorphous solid dispersion of upadacitinib comprising upadacitinib and a magnesium salt.

15. The amorphous solid dispersion of claim 14, wherein the molar ratio of upadacitinib to the magnesium salt is from about 1:0.4 to about 1:1.

16. The amorphous solid dispersion of claim 15, wherein the molar ratio of upadacitinib to the magnesium salt is from about 1:0.4 to about 1:0.6.

17. The amorphous solid dispersion of claim 15, wherein the magnesium salt is magnesium chloride.

18. A pharmaceutical composition comprising the crystalline form of upadacitinib of claim 6 and one or more pharmaceutically acceptable excipients.

19. The pharmaceutical composition of claim 18, wherein the composition is in the form of a capsule or a tablet.

20. The pharmaceutical composition of claim 19, wherein the composition is in the form of an extended-release tablet.

21. A pharmaceutical composition comprising the amorphous solid dispersion of claim 14 and one or more pharmaceutically acceptable excipients.
